SEQUENCE LISTING (2 sequences)

SEQ ID NO: 1 - DNA, 3691 bp, Homo sapiens
  CDS (477)..(3137)
gggccggggtgcggagggagtccctgaaacttctcccaacagccccagggccaatcttccctcagcccactcccctcttcgccagctcccaggtccccactcctgtaccgggggtggggtggggagccaggcccgtctcccgctgggacacacacaggggccggggagcggggacgggacccccgaggcgggggggacgaaccgacagacagacggctgggcgcccgccccagcgggcaggcaggcaggaggaggcggaggcgggggtacgacggggagcactgggtctggggagtttcctctcaactatcgggggagaaactccccgcagccggaggaaagacccagacagtgttttcctcccgggggccgtgctcccccgccccgcgtagcggcggtcgccgccaccggcgcctccacctctaccatctcctctttctccaccacctcgggccccggtgtccccggccagcactatgcccatcttactgttcctgatagacacgtctgcctctatgaaccagcgcagccatctgggcaccacctacctggacacggccaaaggcgcggtagagaccttcatgaagctccgtgcccgggaccctgccagcagaggagacaggtatatgctggtcactttcgaagagccgccctatgctatcaaggctggatggaaagaaaaccatgcaacgtttatgaatgaattgaaaaaccttcaggctgaaggacttacgactcttggccaatccctaaggacagcttttgatttattaaatttaaatagattagtaactggcatagacaactatgggcagggaagaaacccttttttcttggagccagcaataattatcacaattactgatgggagcaagttgactaccaccagtggagtccaggatgagcttcatttacctcttaattctcctttgcctggaagtgaattgaccaaggaaccttttcgttgggatcagagactctttgcattagtgttgcggttgcctggcaccatgtcagtagaatcagaacagttgacaggtgtgcctttagatgactctgcaatcacaccaatgtgtgaagtgacaggcggccgttcatattctgtgtgttctccaagaatgcttaatcagtgtctggagtccttggtgcagaaagtacaaagtggggtggtaataaactttgaaaaagcaggaccagatccttcccctgtagaagatgggcagccagatatatcaaggccttttggatctcagccttggcatagctgtcacaaactcatatatgtcagaccaaatcctaaaactggggttcctataggtcattggcctgttccagagtctttttggccagatcaaaattcgccaacactaccacctcgtacatctcatcctgtagtgaagttttcctgtacagactgtgaaccaatggttattgataaacttccttttgacaaatatgagttggaaccttcaccactgactcaatttatcctggaaaggaaatctcctcaaacatgttggcaggtgtacgtgagcaatagtgcaaaatacagtgaacttggtcatccttttggttacttgaaagccagtacagcactgaactgtgtcaacttatttgtgatgccttacaattatccagtccttcttcccctcttagatgacttgtttaaagtgcataaagcaaaaccaacattgaagtggagacagtcatttgaaagttatttgaagacaatgcctccctactatcttgggcccttgaagaaagctgttaggatgatgggagcacctaacctaatagcagacagtatggaatatggacttagttacagtgtcatttcatacctcaaaaaactgagtcaacaggccaaaatagaatctgatcgagtcattggatctgtaggcaaaaaagtagtacaggagactggaataaaagtccggagccgatcacatggtttatcaatggcatataggaaagattttcaacaactcctccagggaatttcagaggatgtccctcacagactgctagaccttaatatgaaggaatacactgggttccaagttgctttgctgaataaggatttgaagccacagacatttagaaatgcttatgacataccaagacgaaatcttttggatcacttaacaagaatgagatctaatcttttgaagagcactcgcagatttctgaaaggacaggacgaagatcaagtgcacagtgttcctatagcacaaatggggaactaccaggaatacctcaagcaagtaccttctccactaagagaacttgatcctgatcagccacgaaggttgcatacatttggcaacccctttaagctggataagaagggtatgatgatagatgaagcagatgaatttgtggctggacctcaaaataaacataaacgacccggagaaccaaatatgcaagggatccctaaaagacgtcggtgtatgtctccactactaagaggcagacagcagaatcctgttgtaaacaatcatattgggggaaaaggaccacctgcacctacaactcaagcacagccagatcttattaaacctcttcctcttcataaaatttcagaaaccactaatgattcgataatacatgatgtggttgaaaatcatgttgcagaccaactttcatcagacattacaccaaatgctatggatacggaattttcagcatcttctccagccagtttactggaacggccaaccaatcatatggaggctcttggtcatgaccatttaggaaccaatgacctcactgttggtggatttttagaaaatcatgaggagccaagagataaagaacaatgtgctgaagagaacataccagcatcttcactcaacaaaggaaagaaattgatgcattgcagaagccatgaagaggtcaatactgaactaaaagcacaaataatgaaagagatccgaaagccaggaagaaaatatgaaagaatcttcactttactgaagcatgtgcaaggcagtttacaaacaagactaatatttttacaaaatgtcattaaagaagcatcaaggtttaaaaaacgaatgctaatagaacaactggagaacttcttggatgaaattcatcgaagagccaatcagatcaaccatattaatagcaattaaaagaaaatagaatgtggccacttatttcactatcttcttcaaatacaaagtaaataacaagactgttgtgatcttgcattcattttctgacatgcattgttggctatttgaaatactaaaagcaaatctacagatcctttttccatcattttacagtgaccttttcttcattttggtttatttttgtaatgtgaaaagtatcactctaaaaaacatttttaatttaacaaactaaaaatattcctccaaatctcttgcttgtcattgactcttgcgtgtcaatttccctcaggttctattttcttaaaccaacctttaaaattgtcacctctgttaaggttgaactttgccaaaaaaaaaagaagttactttgtaatttttggggaaaaaagcacatacattaaaactaggtaatgttttgtatatacagttattttggatatattattgtaagttgtacaaatgtattttgaagaatatttaagaaaagcacttttgttattccatcaataaatgctctattttaaaaaaaaaaaaaaaaaaaaaaaaaa

SEQ ID NO: 2 - protein, 887 aa, Homo sapiens
MPILLFLIDTSASMNQRSHLGTTYLDTAKGAVETFMKLRARDPASRGDRYMLVTFEEPPYAIKAGWKENHATFMNELKNLQAEGLTTLGQSLRTAFDLLNLNRLVTGIDNYGQGRNPFFLEPAIIITITDGSKLTTTSGVQDELHLPLNSPLPGSELTKEPFRWDQRLFALVLRLPGTMSVESEQLTGVPLDDSAITPMCEVTGGRSYSVCSPRMLNQCLESLVQKVQSGVVINFEKAGPDPSPVEDGQPDISRPFGSQPWHSCHKLIYVRPNPKTGVPIGHWPVPESFWPDQNSPTLPPRTSHPVVKFSCTDCEPMVIDKLPFDKYELEPSPLTQFILERKSPQTCWQVYVSNSAKYSELGHPFGYLKASTALNCVNLFVMPYNYPVLLPLLDDLFKVHKAKPTLKWRQSFESYLKTMPPYYLGPLKKAVRMMGAPNLIADSMEYGLSYSVISYLKKLSQQAKIESDRVIGSVGKKVVQETGIKVRSRSHGLSMAYRKDFQQLLQGISEDVPHRLLDLNMKEYTGFQVALLNKDLKPQTFRNAYDIPRRNLLDHLTRMRSNLLKSTRRFLKGQDEDQVHSVPIAQMGNYQEYLKQVPSPLRELDPDQPRRLHTFGNPFKLDKKGMMIDEADEFVAGPQNKHKRPGEPNMQGIPKRRRCMSPLLRGRQQNPVVNNHIGGKGPPAPTTQAQPDLIKPLPLHKISETTNDSIIHDVVENHVADQLSSDITPNAMDTEFSASSPASLLERPTNHMEALGHDHLGTNDLTVGGFLENHEEPRDKEQCAEENIPASSLNKGKKLMHCRSHEEVNTELKAQIMKEIRKPGRKYERIFTLLKHVQGSLQTRLIFLQNVIKEASRFKKRMLIEQLENFLDEIHRRANQINHINSN

(19) United States
(12) Patent Application Publication
(10) Pub. No.: US 2002/0004232 A1
(43) Pub. Date: Jan. 10, 2002

(54) Tumor suppressor gene del-27

(76) Inventor: Ilse Wieland, Essen (DE)

Correspondence Address: HOFFMANN-LA ROCHE INC., PATENT LAW DEPARTMENT, 340 KINGSLAND STREET, NUTLEY, NJ, 07110

(21) Appl. No.: 09/771,404

(22) Filed: Jan. 26, 2001

Related U.S. Application Data
(63) Continuation of application No. 09/255,947, filed on Feb. 23, 1999, now abandoned.

(30) Foreign Application Priority Data
Feb. 26, 1998 (EP) 98103334.3

(51) Int. Cl. C12Q 1/68; C07H 21/04; C12N 9/00

(52) U.S. Cl. 435/183; 435/6; 536/23.2

(57) ABSTRACT

The invention comprises an isolated nucleic acid molecule having SEQ ID NO:1 and the complementary sequence, as well as nucleic acid molecules hybridizes under stringent conditions to SEQ ID NO:1. SEQ ID NO:1 is a new tumor suppressor gene.

### CROSS REFERENCE TO RELATED APPLICATION

This is a continuation of copending patent Application Ser. No. 09/255,947, filed Feb. 23, 1999.

### FIELD OF INVENTION

The present invention relates to the new tumor suppressor gene del-27, the protein coded thereby, and their use for diagnostics and therapeutics, especially in the field of cancer. In particular, the invention relates to the diagnosis of the lack of tumor suppressor gene del-27 in mammalian, especially in tumor, cells and gene therapy methods to restore del-27 and its functions in mammalian cells, especially in tumor cells.

### BACKGROUND OF THE INVENTION

Tumor suppressor genes are typically thought of as genes whose expression is reduced or lost in cancer cells (Knudson, Proc. Natl. Acad. Sci. USA 19 (1993) 10914-10921). The lack of expression results from mutations in the genes encoding their proteins. Since these proteins are believed to suppress cell growth and thereby act as negative growth regulators, loss of their expression in tumor cells leads to the increased cell proliferation observed and contributes to malignant transformation. As negative growth regulators, tumor suppressor gene products are likely to have also normal functions critical to the development of differentiated tissues. In this respect, tumor suppressor genes may have an important role in the growth arrest necessary for the onset of cellular differentiation as growth regulation is a normal feature of development and differentiation. An overview of tumor suppressor genes is given by, e.g., Gutmann, D. H., Int. J. Dev. Biol. 39 (1995) 895-907.

Inactivation of tumor suppressor genes appears to be a predominant genetic event in the genesis and progression of many tumors. In normal cells, these genes are thought to be involved in the regulation of cell proliferation and differentiation (Fearon, E., and Vogelstein, B., Cell 61 (1990) 759-767). Inactivating mutations and deletions of tumor suppressor genes may therefore release normal growth constraints and may result in the development or progression of tumor cells. A genomic region that contains a putative tumor suppressor gene can be identified by frequent loss of heterozygosity (LOH) of the normal allele with the remaining allele beeing presumably non-functional in the tumor cells (Fearon, E., and Vogelstein, B., Cell 61 (1990) 759-767).

Recently, frequent LOH at the del-27 locus on the short arm of chromosome 5 in human lung carcinomas was demonstrated (Wieland, I., and Böhm, M., Cancer Res. 54 (1994) 1772-1774; Wieland, I., et al., Oncogene 12 (1996) 97-102). Furthermore, LOH at the del-27 locus correlated with tumor progression in transitional cell carcinoma (B\ddot{o}hm, M., Int. J. Cancer 72 (1997) 1-5). del-27 was isolated by genomic difference cloning and is homozygously deleted in a lung carcinoma cell line supporting its close linkage to a novel putative tumor suppressor gene (Wieland, I., Proc. Natl. Acad. Sci. USA 87 (1992) 2720-2724). However, no cDNA coding for a tumor suppressor gene was described therein.

### SUMMARY OF THE INVENTION

It is an object of the invention to provide a novel tumor suppressor gene from the del-27 locus. The invention comprises an isolated nucleic acid molecule (del-27) having the nucleic acid sequence SEQ ID NO:1. The invention also comprises an isolated nucleic acid molecule that hybridizes under stringent conditions with each of nucleic acid fragments 477-819, 820-3069 and 3070-3137 of SEQ ID NO:1, or the complementary nucleic acids therto, and which is capable of suppressing cell proliferation.

The invention further comprises a recombinant polypeptide which suppresses cell proliferation and is selected from the group consisting of

(a) a polynucleotide that is coded by the DNA sequence shown in SEQ ID NO:1.

(b) a polypeptide that is coded by DNA sequences which hybridize under stringent conditions with each of the nucleic acid fragments 477-819, 820-3069 and 3070-3137 of SEQ ID NO:1. or the complementary DNA sequences thereto, and a polypeptide that is coded by DNA sequences which, if there was no degeneracy of the genetic code, would hybridize under stringent conditions with the sequences defined in a) or b) above.

### DETAILED DESCRIPTION OF THE INVENTION

The polypeptides according to the invention, which are described immediately above, can be defined by their DNA sequences as well as by the amino acid sequence derived therefrom.

The del-27 polypeptide can occur in natural allelic variations which differ from individual to individual. Such variations of the amino acids are usually amino acid substitutions. However, they may also be deletions, insertions or additions of amino acids to the total sequence. The del-27 protein according to the invention can be in glycosylated or non-glycosylated form depending on the cell type in which it is expressed and the extent to which it is expressed in a particular cell. Polypeptides with tumor-suppressive activity can easily be identified by a tumor growth inhibition assay using carcinoma cells expressing said polypeptides and measuring the proliferation capacity and apoptosis in relation to carcinoma cells not expressing said polypeptides.

### Definitions

As used herein, the following terms have the following meanings: “Polypeptide with del-27 activity or del-27” means also proteins with minor amino acid variations but with substantially the same del-27 activity. Substantially the same means that the activities are of the same biological properties and the polypeptides show preferably at least 75% homology in amino acid sequence. More preferably, the amino acid sequences are at least 90% identical.

The term “nucleic acid molecule” denotes a polynucleotide which can be, for example, a DNA, RNA, or derivatized active DNA or RNA. DNA and/or RNA molecules are preferred, however.

The term “hybridize under stringent conditions” means that two nucleic acid fragments are capable of hybridization to one another under standard hybridization conditions described in Sambrook et al., “Expression of cloned genes in E. coli” in Molecular Cloning: A laboratory manual (1989) Cold Spring Harbor Laboratory Press, New York, USA, 9.47-9.62 and 11.45 - 11.61.

More preferably, “stringent conditions” as used herein refer to hybridization in 6.0×SSC at about 45° C., followed by a wash of 2.0×SSC at 50° C. For selection of the stringency the salt concentration in the wash step can be selected, for example from about 2.0×SSC at 50° C., for low stringency, to about 0.2×SSC at 50° C., for high stringency. In addition, the temperature in the wash step can be increased from low stringency conditions at room temperatures, about 22° C., to high stringency conditions at about 65° C.

The term “isolated” as used throughout this application refers to a nucleic acid or polypeptide having del-27 activity and being substantially free of cellular material or culture medium, when produced by recombinant DNA techniques, or being substantially free of chemical precursors or other chemicals, when synthesized chemically. An isolated nucleic acid is preferably free of sequences which naturally flank the nucleic acid (i.e. sequences located at the 5′ and the 3′ ends of the nucleic acid) in the organism from which the nucleic acid is derived.

del-27 can be purified after recombinant production by affinity chromatography using known protein purification techniques, including immunoprecipitation, gel filtration, ion exchange chromatography, chromatofocussing, isoelectric focussing, selective precipitation, electrophoresis, and the like.

The polypeptides according to the invention can be produced by recombinant means or synthetically. Non-glycosylated del-27 polypeptide is obtained when it is produced recombinantly in prokaryotes. With the aid of the nucleic acid sequences provided by the invention it is possible to search for the del-27 gene or its variants in genomes of any desired cells (e.g. apart from human cells, also in cells of other mammals), to identify these and to isolate the desired gene coding for the del-27 protein. Such processes and suitable hybridization conditions are known to a person skilled in the art and are described, for example in Sambrook, J., et al., “Expression of cloned genes in E. coli” in Molecular Cloning: A laboratory manual (1989) Cold Spring Harbor Laboratory Press, New York, USA, and B. D. Hames, S. G. Higgins, Nucleic acid hybridisation—a practical approach (1985) IRL Press, Oxford, England. In the current application, the standard protocols described in these publications were usually used for the experiments reported infra.

The use of recombinant DNA technology enables the production of numerous active del-27 derivatives. Such derivatives can, for example, be modified in individual or several amino acids by substitution, deletion or addition. The derivatization can, for example, be carried out by means of site directed mutagenesis. Such variations can be easily carried out by a person skilled in the art (J. Sambrook, B. D. Hames, loc. cit.). It merely has to be ensured by means of the below-mentioned tumor cell growth inhibition assay that the characteristic properties of del-27 are preserved. The invention therefore is also directed to del-27 polypeptides that are the products of a prokaryotic or eukaryotic expression of an exogenous DNA.

With the aid of the disclosed nucleic acids coding for an del-27 protein, the proteins according to the invention can be obtained in a reproducible manner and in large amounts. For expression in prokaryotic or eukaryotic organisms, such as prokaryotic host cells or eukaryotic host cells, the nucleic acid sequence is integrated into suitable expression vectors, according to methods familiar to a person skilled in the art. Such an expression vector preferably contains a regulatable/inducible promoter. These recombinant vectors are then introduced for the expression into suitable host cells such as, e.g., E. coli as a prokaryotic host cell or Saccharomyces cerevisiae, Terato carcinoma cell line PA-I sc 9117 (Büittner et al., Mol. Cell. Biol. 11 (1991) 3573-3583), insect cells, CHO or COS cells as eukaryotic host cells, and the transformed or transduced host cells are cultured under conditions which allow an expression of the heterologous gene. The isolation of the protein can be carried out according to known methods from the host cell or from the culture supernatant of the host cell. Such methods are described for example by Ausubel I., Frederick M., Current Protocols in Mol. Biol. (1992), John Wiley and Sons, New York. Also in vitro reactivation of the protein may be necessary if it is not found in soluble form in the cell culture.

The invention further comprises recombinant expression vectors which are suitable for the expression of del-27 and the above-described related proteins, recombinant host cells transfected with such expression vectors, as well as a process for the recombinant production of a protein which is coded by a tumor suppressor gene del-27.

The invention further comprises a method for detecting a nucleic acid molecule of tumor suppressor gene del-27, comprising incubating a sample (e.g. body fluids such as blood, cell lysates) with the isolated nucleic acid molecule according to the invention and determining hybridization under stringent conditions of said isolated nucleic acid molecule to a target nucleic acid molecule as a determination of presence of a nucleic acid molecule which is the del-27 tumor suppressor gene.

The invention further comprises a method for producing a protein which is capable of suppressing cell proliferation by expressing an exogenous DNA in prokaryotic or eukaryotic host cells and isolation of the desired protein, wherein the DNA

a) is coded by the DNA sequence shown in SEQ ID NO:1, or

b) is coded by DNA sequence which hybridizes under stringent conditions with each of the nucleic acid fragments 477-819, 820-3069 and 3070-3137 of SEQ ID NO:1, or the complementary DNA sequences, thereto, or

c) is coded by a DNA sequence which, if there was no degeneracy of the genetic code, would hybridize under stringent conditions with any of the sequences defined in a) or b) above.

The invention further comprises an isolated protein according to the invention which is encoded by a nucleic acid molecule having the nucleotide sequence set forth in SEQ ID NO:1.

The present invention relates to the cloning and characterization of a tumor suppressor gene which is especially characterized as a tumor progression gene. The functional loss of the tumor suppressor gene according to the invention (del-27) releases contact inhibition and anchorage dependence in tumor cells. Therefore the loss of del-27 gene correlates with a more aggressive behavior of the tumor cells and also the potential of the formation of metastasis.

According to the invention, del-27 and its expression products can be used to inhibit tumor growth, preferably of sporadic tumors (in particular, lung and bladder carcinomas) in vivo, preferably by somatic gene therapy.

In combination with retro- or adenoviral vectors the observed tumor suppressive activity of the del-27 cDNA or products of it can be used to inhibt tumor growth of sporadic tumors (in particular in carcinomas such as lung and bladder carcinomas) in vivo by somatic gene therapy.

### EXAMPLES

The following examples, references and sequence listing are provided to aid the understanding of the present invention, the true scope of which is set forth in the appended claims. It is understood that modifications can be made in the procedures set forth without departing from the spirit of the invention.

### Materials and Methods

Screening and Analysis of Phage Clones:

Phage pools containing 10⁵ plaque forming units produced from normal human genomic libraries (Clontech, Cat.# HL1067J; Stratagene, Cat.# 946205) and human fetal and placental cDNA libraires (Clontech, Cat.# HL1065a and # HL1075b) were screened by the polymerase chain reaction (PCR). PCR conditions were as described in Wieland, I., and Böhm, M., Cancer Res. 54 (1994) 1772-1774. For plaque screening filter lifts were hybridized with 32p-labeled probes and washed under standard conditions as described in Sambrook, J., et al., Molecular Cloning: A Laboratory Manual, Cold Spring Harbor, N.Y. Cold Spring Harbor Laboratory, 1989. Postive phage were rescreened by PCR and plaque lift hybridization. Single positive plaques were isolated and phage DNA was prepared using a kit (Qiagen, Cat.# 12523). Inserts were analysed by restriction enzyme cleavage and Southern blot hybridization. For genomic clones, subdones of the phage inserts were produced by cleavage with SacI (Boehringer Mannheim) and cloning (Sambrook, J., et al., Molecular Cloning: A Laboratory Manual, Cold Spring Harbor, N.Y. Cold Spring Harbor Laboratory, 1989) into the SacI site of plasmid vector pT7T3 (Stratagene). Selection of single-copy fragments was based on agarose gel electrophoresis of restriction enzyme cleaved inserts and counter-selection of repetitive sequences using total genomic DNA as a probe in Southern blot hybridizations (Seltmann, M., et al., Cytogenet. Cell Genet. 67 (1994) 46-51). Genomic Southern blot hybridizations were performed as described (Wieland, I., and Böhm, M., Cancer Res, 54 /1994) 1772-1774; Wieland, I., et al., Proc. Natl. Acad. Sci. USA 87 (1992) 2720-2724).

### DNA Sequencing

Both strands of overlapping cDNA clones were sequenced by a commerically available source (Eurogentec, Seraing, Bel.). DNA sequences were analysed using DNAsis, sequence analysis software (Hitachi).

### Cell Lines and Tumor Specimens

Human lung cancer cell lines were obtained and cultivated as described in Wieland, I., and Böhm, M., Cancer Res, 54 /1994) 1772-1774; Wieland, I., et al., Proc. Natl. Acad. Sci. USA 87 (1992) 2720-2724.

### Example 1

Analysis of the del-27 Genomic Region

For analysis of the region flanking the del-27 sequence normal human genomic phage libraries were screened by PCR for the presence of a 356bp PCR product of the del-27 sequence (Wieland, I., and Böhm, M., Cancer Res, 54 /1994) 1772-1774). Two overlapping phage clones covering 21 kb of the del-27 region were isolated and subcloned into a plasmid vector. Single-copy subclones from the del-27 region were used as hybridization probes on genomic Southern blots. Probe p17E contained an internal HindII site that is hybridized to a 4.3 kb and a second larger HindII fragment. In human lung carcinoma cell line SK-LC-17, however, the 4.3 kb HindII fragment was missing while a smaller fragment of 1.3 kb was apparent. Thus, subdone p17E identified a homozygous deletion of 3 kb in this cell line. This homozygous deletion includes the previously isolated del-27 sequence. A PstI site was also lost resulting in a rearranged PstI restriction fragment. Only 3.5 kb apart from this deletion a second homozygous deletion of 1.4 kb was identified in the same cell line using subclone p13B as a probe. Further homozygous deletions or rearrangements within the 21 kb region were not observed by Southern blotting experiments. This demonstrates that homozygous deletions accompanied by genomic rearrangements occurred within 10 kb of the del-27 region in human lung carcinoma cell line SK-LC-17. Probe p13B produced, in addition to the strong hybridizing signal from the del-27 region on chromosome 5, weaker signals on genomic Southern blots. To determine whether probe p13B cross-hybridized with another chromosomal region in the human genome, a somatic cell hybrid panel was analysed (Wieland, I., et al., Proc. Natl. Acad. Sci. USA 87 (1992) 2720-2724). Cross-hybridizing signals were detected in somatic cell hybrids containing human chromosome 13. This indicates that a sequence homologous to the p13B sequence from the del-27 region exists on human chromosome 13. To investigate whether subclones p17E and p13B detect potential exon sequences, the subclones were analysed for cross-species conservation. Both subclones hybridized to genomic fragments from several species. In particular, subdone p13B appeared to be highly conserved because it hybridized to all mammalian DNAs tested (man, African green monkey, mouse, rat, hamster, dog, cow) and also to chicken. This suggested that exon sequences of a gene exist in the del-27 region.

### Example 2

Isolation of the del-27 cDNA

Human cDNA libraries were screened by PCR using primers deduced from the highly conserved clone p13B. Several overlapping clones were isolated and sequenced. The aligned cDNA of 3665bp contains an open reading frame of 2661bp (from nucleotide postion 477 to 3137). This corresponds to an 887 amino acid sequence. Comparison of the del-27 cDNA with known cDNAs from GeneBank identified a region of homology (with 90% identity) from nucleotide position 820 to 3069 to a mouse cDNA (GenBank accession number

U57368, MMU57368) encoding a putative transmembrane protein involved in contact inhibition and anchorage dependent growth.

These results suggest that functional loss of the del-27 gene and/or related genes releases contact inhibition and anchorage dependence in tumor cells. This is supported by the observation that loss of the del-27 gene correlates with a more aggressive behavior of the tumor cells in bladder carcinoma (Böhm, M., et al., Int. J. Cancer 72 (1997) 1-5).

### Example 3

Functional test of the del-27 cDNA as a Tumor Suppressor Gene

The tumor suppressive activity of the del-27 cDNA can be tested by introduction of either the entire coding region or parts of the coding region of the del-27 cDNA into carcinoma cells:

The entire coding region or in frame parts of the coding region are cloned into the tetracycline-inducible expression vector pUHD10-3 (Gossen, M., and Bujard, H., Proc. Natl. Acad. Sci. USA 89 (1992) 5547-5551). Together with a selection marker the different constructs are then electroporated into carcinoma cells containing the rtTa-gene on plasmid pUHD172-1neo (Gossen, M., et al., Science 268 (1995) 1766-1769). In selected cell clones expression of the del-27 cDNA is induced by treatment of the cells with tetracycline or doxycycline. In vitro, tumor suppressive activity of the del-27 cDNA constructs are determined by measuring the proliferative capacity and apoptosis of induced cells compared to non-induced cells. The generated carcinoma cells are further investigated in an in vivo mouse model. Cells are injected into immune-deficient mice. Expression of the del-27 cDNA constructs in the carcinoma cells is induced by tetracycline when small tumor nodules are apparent. Tumor suppression (tumor growth inhibition, tumor regression) is monitored in the induced and non-induced state.

1. An isolated nucleic acid molecule having the sequence SEQ ID NO:1.

2. An isolated nucleic acid molecule of claim 1 having the sequence 477-819 of SEQ ID No:1.

3. An isolated nucleic acid molecule of claim 1 having the sequence 820 to 3069 of SEQ ID NO:1.

4. An isolated nucleic acid molecule of claim 1 having sequence the 3070 to 3137 of SEQ ID NO:1.

5. An isolated polypeptide having the sequence SEQ ID NO:2.

6. An isolated polypeptide encoded by DNA sequence SEQ ID NO:1.

7. An isolated polypeptide of claim 6 encoded by DNA sequence 477-819 of SEQ ID NO:1.

8. An isolated polypeptide of claim 6 encoded by DNA sequence 820 to 3069 of SEQ ID NO:1.

9. An isolated polypeptide of claim 6 encoded by DNA sequence the 3070 to 3137 of SEQ ID NO:1.

10. A method for determining that a tumor cell lacks metastatic potential by detecting the presence of tumor suppressor gene del-27 in the tumor cell, comprising:
a. contacting a sample containing tumor cell DNA with a nucleic acid probe having the sequence SEQ ID NO:1 under stringent hybridization conditions; and
b. detecting a tumor cell DNA capable of hybridizing to the full length of the probe, thereby determining that the tumor cell lacks metastatic potential.

11. A method of determining that a tumor cell has metastatic potential by detecting the deletion or rearrangement of tumor suppressor gene del-27 in the tumor cell, comprising:
a. contacting a sample containing tumor cell DNA with a nucleic acid probe having the sequence SEQ ID NO:1 under stringent hybridization conditions; and
b. detecting no tumor cell DNA capable of hybridizing to the full length of the probe, thereby determining that the tumor cell has metastatic potential.
